(19) United States
(12) Patent Application Publication
(10) Pub. No.: US 2024/0078842 A1
(43) Pub. Date: Mar. 7, 2024

(54) POSTURE CORRECTION SYSTEM AND METHOD

(71) Applicant: HTC Corporation, Taoyuan City (TW)

(72) Inventors: Jia-Yau SHIAU, Taoyuan City (TW); KuanHsun WANG, Taoyuan City (TW)

(21) Appl. No.: 17/929,314

(22) Filed: Sep. 2, 2022

(51) Int. Cl. G06V 40/20; G06V 10/774; G06V 10/80; G06V 10/82

(52) U.S. Cl. CPC G06V 40/23; G06V 10/7747; G06V 10/803; G06V 10/82

(57) ABSTRACT

A posture correction system and method are provided. The system estimates a body posture tracking corresponding to a user based on a posture image corresponding to the user and a plurality of pressure sensing values, wherein each of the pressure sensing values respectively corresponds to a body part of the user. The system generates a posture adjustment suggestion based on the body posture tracking.

### BACKGROUND

### Field of Invention

The present invention relates to a posture correction system and method. More particularly, the present invention relates to a posture correction system and method capable of assisting a user to adjust posture through a vision and pressure sensing device.

### Description of Related Art

In recent years, the trend of sports has become more and more popular, and people pay more attention to the efficiency and safety of sports. Therefore, it is an inevitable requirement to assist in checking the user's exercise posture through data collection and analysis, so as to improve the user's exercise efficiency.

In the conventional technology, posture analysis is usually performed simply through the image generated by a single camera. However, if the user's posture is analyzed simply based on the image content, it is easy to fail to accurately determine the user's current posture due to the problems of occlusion and scale ambiguity of the image. Therefore, it is difficult to accurately provide the user with a correct posture adjustment suggestion.

Accordingly, there is an urgent need for a posture correction technology that can accurately provide the user with correct posture adjustment suggestions.

### SUMMARY

An objective of the present disclosure is to provide a posture correction system. The posture correction system comprises an image capturing device, a pressure sensing device, and a processing device. The processing device is connected to the image capturing device and the pressure sensing device. The image capturing device is configured to generate a posture image corresponding to a user. The pressure sensing device is configured to detect a plurality of pressure sensing values. The processing device receives the pressure sensing values from the pressure sensing device, wherein each of the pressure sensing values corresponds to a body part of the user. The processing device estimates a body posture tracking corresponding to the user based on the posture image and the pressure sensing values. The processing device generates a posture adjustment suggestion based on the body posture tracking.

Another objective of the present disclosure is to provide a posture correction method, which is adapted for use in an electronic system. The posture correction method comprises following steps: estimating a body posture tracking corresponding to a user based on a posture image corresponding to a user and a plurality of pressure sensing values, wherein each of the pressure sensing values respectively corresponds to a body part of the user; and generating a posture adjustment suggestion based on the body posture tracking.

According to the above descriptions, the posture correction technology (at least including the system and the method) provided by the present disclosure estimates a body posture tracking corresponding to a user based on the posture image corresponding to the user and the pressure sensing values corresponding to each body part of the user. In addition, the posture correction technology provided by the present disclosure generates a posture adjustment suggestion based on the body posture tracking. The posture correction technology provided by the present disclosure can assist the user to adjust the posture through the vision and pressure sensing devices, thus solving the disadvantage that the prior art cannot provide the correct posture adjustment suggestion for the user accurately.

The detailed technology and preferred embodiments implemented for the subject disclosure are described in the following paragraphs accompanying the appended drawings for people skilled in this field to well appreciate the features of the claimed invention.

### BRIEF DESCRIPTION OF THE DRAWINGS

FIG. 1 is a schematic view depicting an applicable scenario of a posture correction system of the first embodiment;

FIG. 2 is a schematic view depicting a schematic diagram of the neural network training operation of some embodiments; and

FIG. 3 is a partial flowchart depicting a posture correction method of the second embodiment.

### DETAILED DESCRIPTION

In the following description, a posture correction system and method according to the present disclosure will be explained with reference to embodiments thereof. However, these embodiments are not intended to limit the present disclosure to any environment, applications, or implementations described in these embodiments. Therefore, description of these embodiments is only for purpose of illustration rather than to limit the present disclosure. It shall be appreciated that, in the following embodiments and the attached drawings, elements unrelated to the present disclosure are omitted from depiction. In addition, dimensions of individual elements and dimensional relationships among individual elements in the attached drawings are provided only for illustration but not to limit the scope of the present disclosure.

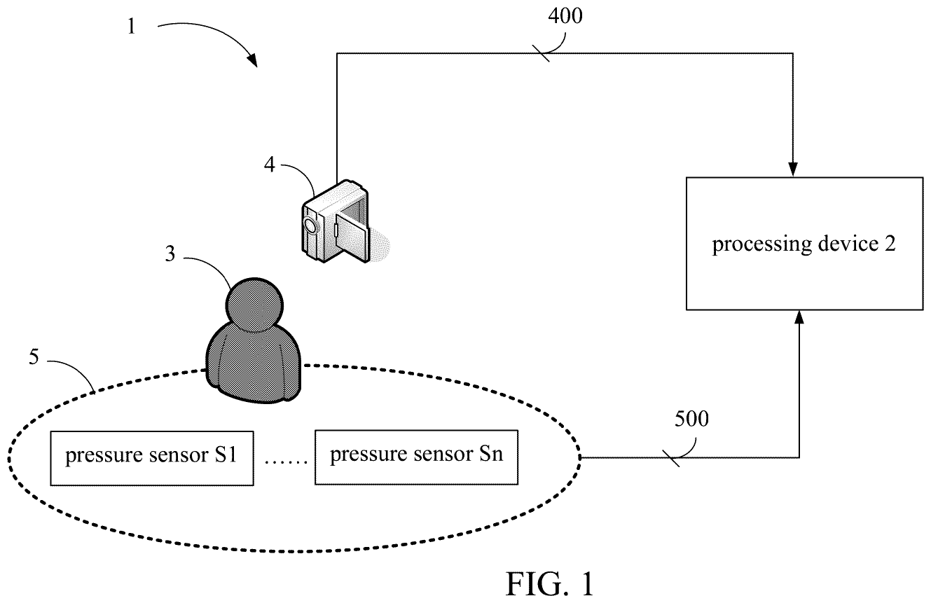

First, an applicable scenario of the present embodiment will be explained, and a schematic view is depicted in FIG. 1. As shown in FIG. 1, in the first embodiment of the present disclosure, the posture correction system 1 comprises a processing device 2, an image capturing device 4, and a pressure sensing device 5. The processing device 2 is connected to the image capturing device 4 and the pressure sensing device 5. The image capturing device 4 can be any device having an image capturing function. The processing device 2 may be any of various processors, Central Processing Units (CPUs), microprocessors, digital signal processors or other computing apparatuses known to those of ordinary skill in the art.

In this scenario, the user 3 uses the object provided with the pressure sensing device 5 to perform an action or exercise. Specifically, the pressure sensing device 5 comprises a plurality of pressure sensors S1, . . . , Sn, and the pressure sensors S1, . . . , Sn are configured to detect a plurality of pressure sensing values 500, where n is a positive integer greater than 2. For example, the object provided with the pressure sensing device 5 may be a pad (e.g., a yoga pad), a sportswear, a sports pant, tights, a grip, a bat, a steering wheel, and the like.

It shall be appreciated that the processing device 2 may be connected to the pressure sensing device 5 through a wired network or a wireless network. The pressure sensors S1, . . . , Sn are used to continuously generate the pressure sensing values 500 (e.g., at a frequency of 10 times per second), and the pressure sensing device 5 transmits the pressure sensing values 500 to the processing device 2.

It shall be appreciated that each of the pressure sensing values 500 generated by the pressure sensors S1, . . . , Sn may correspond to a body part of the user 3 (e.g., joints, etc). For example, if the object used by the user 3 is the sports pant, the pressure sensing device 5 can set the pressure sensors S1, . . . , Sn on the sports pant corresponding to the thigh, calve, knee joint, ankle joint, and hip joint and other body parts for data collection. For another example, if the object used by the user 3 is a yoga pad, the pressure sensing device 5 can evenly arrange the pressure sensors on the yoga pad to collect data on the body parts of the user 3 touching the yoga pad.

As shown in FIG. 1, the image capturing device 4 can be installed near the user 3 to facilitate capturing a posture image of the user 3. The processing device 2 can be connected to the image capturing device 4 through a wired network or a wireless network. The image capturing device 4 is configured to generate a posture image 400 corresponding to the user 3, and transmit the posture image 400 to the processing device 2. The posture image 400 can record the current posture of the user 3.

In the present embodiment, the image capturing device 4 may comprise one or a plurality of image capturing units (e.g., one or a plurality of depth camera lenses) for generating the posture image 400 corresponding to a field of view.

In some embodiments, the image capturing device 4 and the processing device 2 may be located in the same device. Specifically, the image capturing device 4 and the processing device 2 can be comprised in an all-in-one (AIO) device, and the all-in-one device is connected to the pressure sensing device 5. For example, the all-in-one device may be a mobile phone with a computing function and image capturing function.

It shall be appreciated that, FIG. 1 is only used as an example, and the present disclosure does not limit the content of the posture correction system 1. For example, the present disclosure does not limit the number of device connected to the processing device 2. The processing device 2 can simultaneously connect to multiple pressure sensing devices and multiple image capturing devices through the network, and it depends on the scale and actual requirements of the posture correction system 1.

In the present embodiment, the processing device 2 receives the pressure sensing values 500 from the pressure sensing device 5, wherein each of the pressure sensing values 500 corresponds to a body part of the user 3, respectively.

Next, the processing device 2 estimates a body posture tracking corresponding to the user 3 based on the posture image 400 and the pressure sensing values 500. Finally, the processing device 2 generates a posture adjustment suggestion based on the body posture tracking.

In some embodiments, the processing device 2 determines the required posture adjustment by comparing the difference between the body posture tracking and a standard posture. Specifically, the processing device 2 compares the body posture tracking with a standard posture to calculate a posture difference value. Next, the processing device 2 generates the posture adjustment suggestion based on the posture difference value.

For example, the processing device 2 may first determine the standard posture currently corresponding to the body posture tracking. For example, the processing device 2 determines, based on the current body posture tracking, that the movement currently performed by the user 3 should be a Warrior II movement (i.e., one of the yoga movements), and the standard standing posture should be that the left and right feet present 90 degrees. The current determining result shows that the left and right feet of the user 3 are only 75 degrees, so the processing device 2 may remind the user 3 to adjust the left and right feet to 90 degrees.

In some embodiments, in order to make the positioning of the posture more accurate when the processing device 2 analyzes the posture image 400, the processing device 2 can further determine the position of each body part of the user 3 in the space based on the depth information of the posture image 400. Specifically, the processing device 2 analyzes the posture image 400 to generate a spatial position corresponding to each of the body parts of the user 3. Next, the processing device 2 estimates the body posture tracking corresponding to the user 3 based on the spatial positions and the pressure sensing values 500.

In some embodiments, the processing device 2 can estimate the body pose tracking through a fusion analysis neural network. Specifically, the processing device 2 inputs the posture image 400 and the pressure sensing values 500 into a fusion analysis neural network to estimate the body posture tracking corresponding to the user 3, and the fusion analysis neural network is trained based on a pressure analysis neural network and a vision analysis neural network.

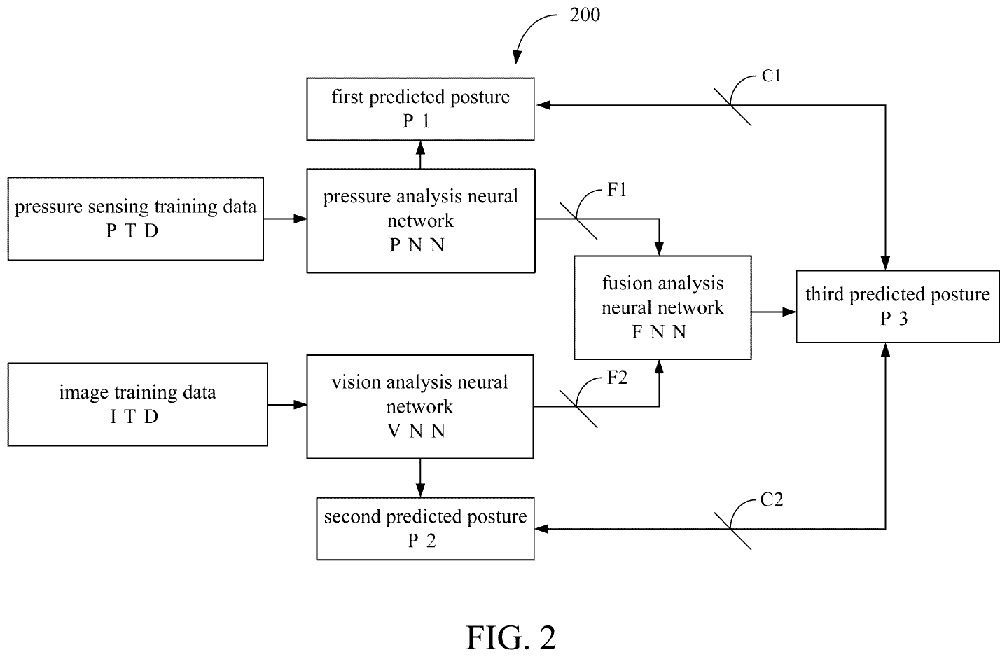

For ease of understanding, the following paragraphs will describe the neural network training method in the present disclosure in detail, please refer to the schematic diagram 200 of the neural network training operation in FIG. 2.

In some embodiments, the processing device 2 may train the pressure analysis neural network PNN based on the labeled pressure sensing training data PTD. Specifically, the processing device 2 collects a plurality of first pressure sensing training data PTD and a first label information (not shown) corresponding to the first pressure sensing training data PTD. Next, the processing device 2 trains the pressure analysis neural network PNN based on the first pressure sensing training data PTD and the first label information.

In some embodiments, the pressure sensing training data PTD may be a synthesis data.

In some embodiments, the processing device 2 may train the vision analysis neural network VNN based on the labeled image training data ITD. Specifically, the processing device 2 collects a plurality of first image training data ITD and a second label information (not shown) corresponding to the first image training data ITD. Next, the processing device 2 trains the vision analysis neural network VNN based on the first image training data ITD and the second label information.

In some embodiments, the processing device 2 may train the fusion analysis neural network FNN based on the labeled paired training data (i.e., including the pressure sensing training data PTD and the image training data ITD). Specifically, the processing device 2 collects a plurality of first paired training data and a third label information (not shown) corresponding to the first paired training data, wherein each of the first paired training data comprises a second pressure sensing training data PTD and a second image training data ITD. Next, the processing device 2 trains the fusion analysis neural network FNN and fine-tunes the pressure analysis neural network PNN and the vision analysis neural network VNNs based on the first paired training data and the third label information.

It shall be appreciated that the processing device 2 can perform the training and fine-tuning operation of the fusion analysis neural network FNN, the pressure analysis neural network PNN, and the vision analysis neural network VNN by calculating the latent feature F1 of the pressure analysis neural network PNN and the latent feature F2 of the vision analysis neural network VNN. Those of ordinary skill in the art shall appreciate the implementation of the neural network training and the fine-tuning operation based on the foregoing descriptions. Therefore, the details will not be repeated herein.

In some embodiments, the processing device 2 may also train the fusion analysis neural network FNN through the unlabeled paired training data and a consistency loss function. Specifically, the processing device 2 collects a plurality of second paired training data, wherein each of the second paired training data comprises a third pressure sensing training data PTD and a third image training data ITD. Next, the processing device 2 calculates the corresponding consistency loss functions C1 and C2 of the pressure analysis neural network PNN and the vision analysis neural network VNN based on the second paired training data. Finally, the processing device 2 trains the fusion analysis neural network FNN and fine-tunes the pressure analysis neural network PNN and the vision analysis neural network VNN based on the second paired training data and the consistency loss functions C1 and C2.

In some embodiments, the processing device 2 calculates the consistency loss function C1 corresponding to the pressure analysis neural network PNN based on a first predicted posture P1 generated by the pressure analysis neural network PNN and a third predicted posture P3 generated by the fusion analysis neural network FNN. In addition, the processing device 2 calculates the consistency loss function C2 corresponding to the vision analysis neural network VNN based on a second predicted posture P2 generated by the vision analysis neural network VNN and a third predicted posture P3 generated by the fusion analysis neural network FNN.

According to the above descriptions, the posture correction system 1 provided by the present disclosure estimates a body posture tracking corresponding to a user based on the posture image corresponding to the user and the pressure sensing values corresponding to each body part of the user. In addition, the posture correction system 1 provided by the present disclosure generates a posture adjustment suggestion based on the body posture tracking. The posture correction system 1 provided by the present disclosure can assist the user to adjust the posture through the vision and pressure sensing devices, thus solving the disadvantage that the prior art cannot provide the correct posture adjustment suggestion for the user accurately.

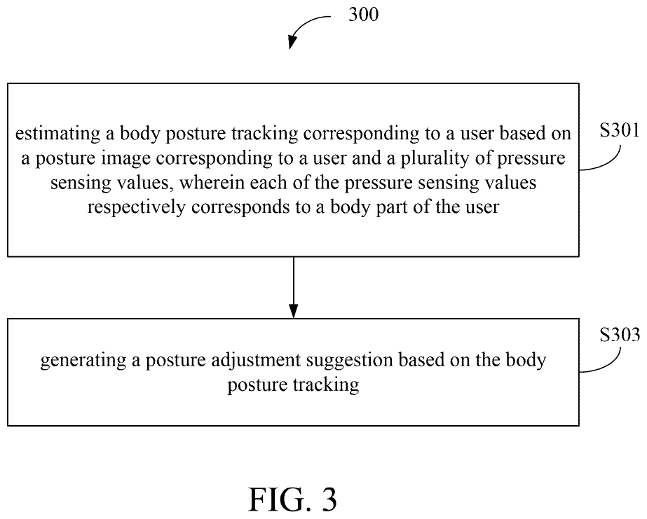

A second embodiment of the present disclosure is a posture correction method and a flowchart thereof is depicted in FIG. 3. The posture correction method 300 is adapted for an electronic system (e.g., the posture correction system 1 of the first embodiment). The posture correction method 300 generates a posture adjustment suggestion through the steps S301 to S303.

In the step S301, the electronic system estimates a body posture tracking corresponding to a user based on a posture image corresponding to a user and a plurality of pressure sensing values, wherein each of the pressure sensing values respectively corresponds to a body part of the user. Next, in the step S303, the electronic system generates a posture adjustment suggestion based on the body posture tracking.

In some embodiments, the posture correction method 300 further comprises following steps: analyzing the posture image to generate a spatial position corresponding to each of the body parts of the user; and estimating the body posture tracking corresponding to the user based on the spatial positions and the pressure sensing values.

In some embodiments, the electronic system comprises a pressure sensing device and an all-in-one device, and the all-in-one device comprises an image capturing device and a processing device (e.g., the processing device 2, the image capturing device 4, and the pressure sensing device 5 of the first embodiment).

In some embodiments, the posture correction method 300 further comprises following steps: inputting the posture image and the pressure sensing values into a fusion analysis neural network to estimate the body posture tracking corresponding to the user; wherein the fusion analysis neural network is trained based on a pressure analysis neural network and a vision analysis neural network.

In some embodiments, the posture correction method 300 further comprises following steps: collecting a plurality of first pressure sensing training data and a first label information corresponding to the first pressure sensing training data; and training the pressure analysis neural network based on the first pressure sensing training data and the first label information.

In some embodiments, the posture correction method 300 further comprises following steps: collecting a plurality of first image training data and a second label information corresponding to the first image training data; and training the vision analysis neural network based on the first image training data and the second label information.

In some embodiments, the posture correction method 300 further comprises following steps: collecting a plurality of first paired training data and a third label information corresponding to the first paired training data, wherein each of the first paired training data comprises a second pressure sensing training data and a second image training data; and training the fusion analysis neural network and fine-tuning the pressure analysis neural network and the vision analysis neural network based on the first paired training data and the third label information.

In some embodiments, the posture correction method 300 further comprises following steps: collecting a plurality of second paired training data, wherein each of the second paired training data comprises a third pressure sensing training data and a third image training data; calculating a consistency loss function corresponding to each of the pressure analysis neural network and the vision analysis neural network based on the second paired training data; and training the fusion analysis neural network and fine-tuning the pressure analysis neural network and the vision analysis neural network based on the second paired training data and the consistency loss functions.

In some embodiments, the posture correction method 300 further comprises following steps: calculating the consistency loss function corresponding to the pressure analysis neural network based on a first predicted posture generated by the pressure analysis neural network and a third predicted posture generated by the fusion analysis neural network; and calculating the consistency loss function corresponding to the vision analysis neural network based on a second predicted posture generated by the vision analysis neural network and a third predicted posture generated by the fusion analysis neural network.

In some embodiments, the posture correction method 300 further comprises following steps: comparing the body posture tracking with a standard posture to calculate a posture difference value; and generating the posture adjustment suggestion based on the posture difference value.

In addition to the aforesaid steps, the second embodiment can also execute all the operations and steps of the posture correction system 1 set forth in the first embodiment, have the same functions, and deliver the same technical effects as the first embodiment. How the second embodiment executes these operations and steps, has the same functions, and delivers the same technical effects will be readily appreciated by those of ordinary skill in the art based on the explanation of the first embodiment. Therefore, the details will not be repeated herein.

It shall be appreciated that in the specification and the claims of the present disclosure, some words (e.g., pressure sensing training data, label information, image training data, paired training data, and predicted posture, etc.) are preceded by terms such as “first”, “second”, and “third”, and these terms of “first”, “second”, and “third” are only used to distinguish these different words. For example, the “first” and “second” label information are only used to indicate the label information used in different operations.

According to the above descriptions, the posture correction technology (at least including the system and the method) provided by the present disclosure estimates a body posture tracking corresponding to a user based on the posture image corresponding to the user and the pressure sensing values corresponding to each body part of the user. In addition, the posture correction technology provided by the present disclosure generates a posture adjustment suggestion based on the body posture tracking. The posture correction technology provided by the present disclosure can assist the user to adjust the posture through the vision and pressure sensing devices, thus solving the disadvantage that the prior art cannot provide the correct posture adjustment suggestion for the user accurately.

The above disclosure is related to the detailed technical contents and inventive features thereof. People skilled in this field may proceed with a variety of modifications and replacements based on the disclosures and suggestions of the disclosure as described without departing from the characteristics thereof. Nevertheless, although such modifications and replacements are not fully disclosed in the above descriptions, they have substantially been covered in the following claims as appended.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A posture correction system, comprising:
an image capturing device, being configured to generate a posture image corresponding to a user;
a pressure sensing device, being configured to detect a plurality of pressure sensing values; and
a processing device, being connected to the image capturing device and the pressure sensing device, and being configured to perform operations comprising:
receiving the pressure sensing values from the pressure sensing device, wherein each of the pressure sensing values corresponds to a body part of the user;
estimating a body posture tracking corresponding to the user based on the posture image and the pressure sensing values; and
generating a posture adjustment suggestion based on the body posture tracking.

2. The posture correction system of claim 1, wherein the processing device is further configured to perform following operations:
analyzing the posture image to generate a spatial position corresponding to each of the body parts of the user; and
estimating the body posture tracking corresponding to the user based on the spatial positions and the pressure sensing values.

3. The posture correction system of claim 1, wherein the image capturing device and the processing device are comprised in an all-in-one device, and the all-in-one device is connected to the pressure sensing device.

4. The posture correction system of claim 1, wherein the processing device is further configured to perform following operations:
inputting the posture image and the pressure sensing values into a fusion analysis neural network to estimate the body posture tracking corresponding to the user;
wherein the fusion analysis neural network is trained based on a pressure analysis neural network and a vision analysis neural network.

5. The posture correction system of claim 4, wherein the processing device is further configured to perform following operations:
collecting a plurality of first pressure sensing training data and a first label information corresponding to the first pressure sensing training data; and
training the pressure analysis neural network based on the first pressure sensing training data and the first label information.

6. The posture correction system of claim 5, wherein the processing device is further configured to perform following operations:
collecting a plurality of first image training data and a second label information corresponding to the first image training data; and
training the vision analysis neural network based on the first image training data and the second label information.

7. The posture correction system of claim 6, wherein the processing device is further configured to perform following operations:
collecting a plurality of first paired training data and a third label information corresponding to the first paired training data, wherein each of the first paired training data comprises a second pressure sensing training data and a second image training data; and
training the fusion analysis neural network and fine-tuning the pressure analysis neural network and the vision analysis neural network based on the first paired training data and the third label information.

8. The posture correction system of claim 6, wherein the processing device is further configured to perform following operations:
collecting a plurality of second paired training data, wherein each of the second paired training data comprises a third pressure sensing training data and a third image training data;
calculating a consistency loss function corresponding to each of the pressure analysis neural network and the vision analysis neural network based on the second paired training data; and
training the fusion analysis neural network and fine-tuning the pressure analysis neural network and the vision analysis neural network based on the second paired training data and the consistency loss functions.

9. The posture correction system of claim 8, wherein the processing device is further configured to perform following operations:
calculating the consistency loss function corresponding to the pressure analysis neural network based on a first predicted posture generated by the pressure analysis neural network and a third predicted posture generated by the fusion analysis neural network; and
calculating the consistency loss function corresponding to the vision analysis neural network based on a second predicted posture generated by the vision analysis neural network and a third predicted posture generated by the fusion analysis neural network.

10. The posture correction system of claim 1, wherein the processing device is further configured to perform following operations:
comparing the body posture tracking with a standard posture to calculate a posture difference value; and
generating the posture adjustment suggestion based on the posture difference value.

11. A posture correction method, being adapted for use in an electronic system, wherein the posture correction method comprises:
estimating a body posture tracking corresponding to a user based on a posture image corresponding to a user and a plurality of pressure sensing values, wherein each of the pressure sensing values respectively corresponds to a body part of the user; and
generating a posture adjustment suggestion based on the body posture tracking.

12. The posture correction method of claim 11, wherein the posture correction method further comprises following steps:
analyzing the posture image to generate a spatial position corresponding to each of the body parts of the user; and
estimating the body posture tracking corresponding to the user based on the spatial positions and the pressure sensing values.

13. The posture correction method of claim 11, wherein the electronic system comprises a pressure sensing device and an all-in-one device, and the all-in-one device comprises an image capturing device and a processing device.

14. The posture correction method of claim 11, wherein the posture correction method further comprises following steps:
inputting the posture image and the pressure sensing values into a fusion analysis neural network to estimate the body posture tracking corresponding to the user;
wherein the fusion analysis neural network is trained based on a pressure analysis neural network and a vision analysis neural network.

15. The posture correction method of claim 14, wherein the posture correction method further comprises following steps:
collecting a plurality of first pressure sensing training data and a first label information corresponding to the first pressure sensing training data; and
training the pressure analysis neural network based on the first pressure sensing training data and the first label information.

16. The posture correction method of claim 15, wherein the posture correction method further comprises following steps:
collecting a plurality of first image training data and a second label information corresponding to the first image training data; and
training the vision analysis neural network based on the first image training data and the second label information.

17. The posture correction method of claim 16, wherein the posture correction method further comprises following steps:
collecting a plurality of first paired training data and a third label information corresponding to the first paired training data, wherein each of the first paired training data comprises a second pressure sensing training data and a second image training data; and
training the fusion analysis neural network and fine-tuning the pressure analysis neural network and the vision analysis neural network based on the first paired training data and the third label information.

18. The posture correction method of claim 16, wherein the posture correction method further comprises following steps:
collecting a plurality of second paired training data, wherein each of the second paired training data comprises a third pressure sensing training data and a third image training data;
calculating a consistency loss function corresponding to each of the pressure analysis neural network and the vision analysis neural network based on the second paired training data; and
training the fusion analysis neural network and fine-tuning the pressure analysis neural network and the vision analysis neural network based on the second paired training data and the consistency loss functions.

19. The posture correction method of claim 18, wherein the posture correction method further comprises following steps:
calculating the consistency loss function corresponding to the pressure analysis neural network based on a first predicted posture generated by the pressure analysis neural network and a third predicted posture generated by the fusion analysis neural network; and
calculating the consistency loss function corresponding to the vision analysis neural network based on a second predicted posture generated by the vision analysis neural network and a third predicted posture generated by the fusion analysis neural network.

20. The posture correction method of claim 11, wherein the posture correction method further comprises following steps:
comparing the body posture tracking with a standard posture to calculate a posture difference value; and
generating the posture adjustment suggestion based on the posture difference value.
